SEQUENCE LISTING (2 sequences)

SEQ ID NO: 1 - protein, 984 aa, Homo sapiens
MALDYLLLLLLASAVAAMEETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPSIVQNFAVFPETMTGAESTSLVIARGTCIPNAEEVDVPIKLYCNGDGEWMVPIGRCTCKPGYEPENSVACKACPAGTFKASQEAEGCSHCPSNSRSPAEASPICTCRTGYYRADFDPPEVACTSVPSGPRNVISIVNETSIILEWHPPRETGGRDDVTYNIICKKCRADRRSCSRCDDNVEFVPRQLGLTECRVSISSLWAHTPYTFDIQAINGVSSKSPFPPQHVSVNITTNQAAPSTVPIMHQVSATMRSITLSWPQPEQPNGIILDYEIRYYEKEHNEFNSSMARSQTNTARIDGLRPGMVYVVQVRARTVAGYGKFSGKMCFQTLTDDDYKSELREQLPLIAGSAAAGVVFVVSLVAISIVCSRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 2 - DNA, 3865 bp, Homo sapiens
cacatgcacacccacacccacgcgcgcccgcaccgccccacgcgcacacactcctgcccacgcccacgcagcgctccgggaagtccggtccgggcgagagcgcgaaaggataccgagaagccacccgcggagagcgcagcggcgccctgggacgcggcgctctcccggcgctgctgcctcggcttggtctcggcctgcgggccgtcggccggcgatggccctggattatctactactgctcctcctggcatccgcagtggctgcgatggaagaaacgttaatggacaccagaacggctactgcagagctgggctggacggccaatcctgcgtccgggtgggaagaagtcagtggctacgatgaaaacctgaacaccatccgcacctaccaggtgtgcaatgtcttcgagcccaaccagaacaattggctgctcaccaccttcatcaaccggcggggggcccatcgcatctacacagagatgcgcttcactgtgagagactgcagcagcctccctaatgtcccaggatcctgcaaggagaccttcaacttgtattactatgagactgactctgtcattgccaccaagaagtcagccttctggtctgaggccccctacctcaaagtagacaccattgctgcagatgagagcttctcccaggtggactttgggggaaggctgatgaaggtaaacacagaagtcaggagctttgggcctcttactcggaatggtttttacctcgcttttcaggattatggagcctgtatgtctcttctttctgtccgtgtcttcttcaaaaagtgtcccagcattgtgcaaaattttgcagtgtttccagagactatgacaggggcagagagcacatctctggtgattgctcggggcacatgcatccccaacgcagaggaagtggacgtgcccatcaaactctactgcaacggggatggggaatggatggtgcctattgggcgatgcacctgcaagcctggctatgagcctgagaacagcgtggcatgcaaggcttgccctgcagggacattcaaggccagccaggaagctgaaggctgctcccactgcccctccaacagccgctcccctgcagaggcgtctcccatctgcacctgtcggaccggttattaccgagcggactttgaccctccagaagtggcatgcactagcgtcccatcaggtccccgcaatgttatctccatcgtcaatgagacgtccatcattctggagtggcaccctccaagggagacaggtgggcgggatgatgtgacctacaacatcatctgcaaaaagtgccgggcagaccgccggagctgctcccgctgtgacgacaatgtggagtttgtgcccaggcagctgggcctgacggagtgccgcgtctccatcagcagcctgtgggcccacaccccctacacctttgacatccaggccatcaatggagtctccagcaagagtcccttccccccacagcacgtctctgtcaacatcaccacaaaccaagccgccccctccaccgttcccatcatgcaccaagtcagtgccactatgaggagcatcaccttgtcatggccacagccggagcagcccaatggcatcatcctggactatgagatccggtactatgagaaggaacacaatgagttcaactcctccatggccaggagtcagaccaacacagcaaggattgatgggctgcggcctggcatggtatatgtggtacaggtgcgtgcccgcactgttgctggctacggcaagttcagtggcaagatgtgcttccagactctgactgacgatgattacaagtcagagctgagggagcagctgcccctgattgctggctcggcagcggccggggtcgtgttcgttgtgtccttggtggccatctctatcgtctgtagcaggaaacgggcttatagcaaagaggctgtgtacagcgataagctccagcattacagcacaggccgaggctccccagggatgaagatctacattgaccccttcacttatgaggatcccaacgaagctgtccgggagtttgccaaggagattgatgtatcttttgtgaaaattgaagaggtcatcggagcaggggagtttggagaagtgtacaaggggcgtttgaaactgccaggcaagagggaaatctacgtggccatcaagaccctgaaggcagggtactcggagaagcagcgtcgggactttctgagtgaggcgagcatcatgggccagttcgaccatcctaacatcattcgcctggagggtgtggtcaccaagagtcggcctgtcatgatcatcacagagttcatggagaatggtgcattggattctttcctcaggcaaaatgacgggcagttcaccgtgatccagcttgtgggtatgctcaggggcatcgctgctggcatgaagtacctggctgagatgaattatgtgcatcgggacctggctgctaggaacattctggtcaacagtaacctggtgtgcaaggtgtccgactttggcctctcccgctacctccaggatgacacctcagatcccacctacaccagctccttgggagggaagatccctgtgagatggacagctccagaggccatcgcctaccgcaagttcacttcagccagcgacgtttggagctatgggatcgtcatgtgggaagtcatgtcatttggagagagaccctattgggatatgtccaaccaagatgtcatcaatgccatcgagcaggactaccggctgcccccacccatggactgtccagctgctctacaccagctcatgctggactgttggcagaaggaccggaacagccggccccggtttgcggagattgtcaacaccctagataagatgatccggaacccggcaagtctcaagactgtggcaaccatcaccgccgtgccttcccagcccctgctcgaccgctccatcccagacttcacggcctttaccaccgtggatgactggctcagcgccatcaaaatggtccagtacagggacagcttcctcactgctggcttcacctccctccagctggtcacccagatgacatcagaagacctcctgagaataggcatcaccttggcaggccatcagaagaagatcctgaacagcattcattctatgagggtccagataagtcagtcaccaacggcaatggcatgagaactcttgtttcttggggaaggagaggagggaaaaggaccagggtcaagggggaccagaggttgaccactgtggaatgtactggagagactggcttctcagctgaggaatgcatttccatcagtgaagaatcaaccggacctgttgctagcaggcaatctccatttctcagtgacagaagcatgtttgagatgccgtgggaaaccaaatatataataataaaaatataaaaaggtgatgttcaacagaagtgaagacaaaacaatatgcatcaggagaacaagagtaaacccagctcccactctcagtgggctgcagttgcccaaccacaggaagaaagggaaggaggtagagggaagaaacagaagcagtgttccattttcttcctcaccaatgacattcttttcttttctcctttcgtactcctccctgagagtcccctcccttctcccacactcgtttccctttgctcatgactcctgtagggaagtttcttcaaacaaaacccagctcctgagtctccagatgttgttctgtcagttgccaaaggactttgctgaccactgcatggggatccaaccaattcaattaatgtcttcatattgaagaagagatgtaccttcaattgaaaacctcgtttttcttttgtttgcattttctgcaaaaaggaaaaagaaaccacaaattggggaattc

(19) United States
(12) Patent Application Publication
(10) Pub. No.: US 2007/0280885 A1
(43) Pub. Date: Dec. 6, 2007

(54) Methods Of Treating Demyelinating Disorders

(75) Inventors: Karen Chandross, Somerset, NJ (US); Jean Merrill, Morristown, NJ (US); Sridaran Natesan, Ashland, MA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Aventis Pharmaceuticals Inc., Bridgewater, NJ (US)

(21) Appl. No.: 11/568,525

(22) PCT Filed: May 4, 2005
(86) PCT No.: PCT/US05/15387

§ 371 Date: Aug. 1, 2007

Related U.S. Application Data
(60) Provisional application No. 60/568,732, filed on May 6, 2004.
(60) Provisional application No. 60/573,915, filed on May 24, 2004.

(51) Int. Cl. C12Q 1/02; C12Q 1/48

(52) U.S. Cl. 424/9.2; 435/15; 435/29

(57) ABSTRACT

Methods of identifying and using compounds capable of treating demyelinating disorders such as multiple sclerosis by inhibiting EphB1-mediated cell repulsion of CNS and PNS glial cells (oligodendrocytes and Schwann cells and progenitor cells within these lineages).

### BACKGROUND OF THE INVENTION

1. Field of the Invention

This invention relates to the field of treating multiple sclerosis and, in particular, to treating multiple sclerosis by inhibiting EphB1-meditated cell repulsion on CNS and peripheral glial cells (olgiodendrocyte and Schwann cell) and progenitor cells within these lineages.

2. Description of the Related Art

All documents cited are incorporated herein in their entirety by reference.

The erythropoietin-producing hepatocellular (“Eph”) receptors are a family of receptor tyrosine kinases containing an extracellular region with a unique cysteine-rich motif and two fibronectin type III motifs, (Connor R J, and Pasquale E B. (1995) Oncogene 11:2429-381995), along with an intracellular tyrosine kinase domain involved in signal transduction. (Vindis, et al., J Cell Biol. Aug. 18, 2003; 162(4): 661-71). The Eph receptors are implicated in neural development and physiology, and are expressed in the developing and adult nervous system. (Tuzi N L and Gullick W J. (1994) Br. J. Cancer 69:417-21). The ligands to the Eph receptors are known as ephrins. All known ephrin ligands are membrane-associated. The ephrin-A subclass is associated to the membrane through a glycosyl phosphatidylinositol (“GPI”) group. The ephrin-B subclass is associated through a transmembrane domain. (Flanagan and Vanderhaeghen, Annu. Rev. Neurosci. 1998. 21:309-45). The ephrin ligands interact with their Eph receptors by direct cell-cell contact (Davis, S., et al. (1994) Science 266, 816-819; Drescher, U., et al. (1997) Curr. Opin. Neurobiol. 7, 75-80; Flanagan, J. G. and Vanderhaeghen, P. (1998) Annu. Rev. Neurosci. 21, 309-345; Frisen, J., et al., (1999) EMBO J. 18, 5159-5165; Mellitzer, G., et al., (1999) Nature 400, 77-81).

The ephrin ligands have been shown to act as repulsive axon guidance cues, and Eph receptors are required for correct axonal navigation in vivo. (Holland, S. J., et al., (1998) Curr. Opin. Neurobiol 8, 117-127). The EphB1 receptor tyrosine kinase (“EphB1”), also known as Elk, Cek6, Net, and Hek6, plays a prominent role during central and peripheral nervous system development by establishing proper spatial patterning. Interaction of the EphB1 receptor and ephrin-B ligands has been shown to play a role in neural development. (Smith, et al., Curr Biol. Aug. 1, 1997;7(8):561-70). For example, EphB1 and ephrin-B2 are expressed in complementary patterns in the midbrain dopaminergic neurons and their targets, which suggests that their interaction may contribute to the establishment of distinct neural pathways. (Yue, et al., J Neurosci. Mar 15, 1999;19(6):2090-101.) EphB has been shown to play a role in synapse formation, (Dalva et al. (2000) Cell 103:945.), as well as cell migration and proliferation. (Conover et al. (2000) Nature Neurosci 3:1091). Eph receptors and ephrin ligands have been linked to cell signaling pathways related to cell motility, corroborating their role in cell migration and repulsion. (Schmucker and Zipursky (2001) Cell 105:701). Ephrin B1 is expressed on neuroepithelial cells in correlation with neocortical neurogenesis. (Stuckmann et al. (2001) JNS 21:2726). In addition to neuronal development, Ephs and ephrins have been shown to function in the adult CNS. For example, EphB1 and ephrin-B interactions have been shown to modulate synaptic efficiency and pain processing in the spinal cord. (Battaglia et al. (2003) Nature Neurosci 6:339).

Because both the Eph family and the ephrin family are membrane-associated and communicate with each other through direct cell-cell interaction, designation of the Eph family as “receptors” and the ephrins as “ligands” is somewhat arbitrary. In fact, it has been shown that signaling between the Ephs and the ephrins can be bi-directional. Interaction with an Eph receptor causes an ephrin-B ligand to become tyrosine-phosphorylated and transduce intracellular signals that lead to reorganization of the cytoskeleton of the ephrin-B-expressing cell. (Xu, et al., J. Biol. Chem., 2003, Vol. 278, Issue 27, 24767-24775). It has been shown that ephrin-B transduces the reverse signaling pathway using the Grb4 protein, which is a known adaptor protein for the SH2/SH3 domain. (Cowan C A, and Henkemeyer M., Nature. Sep. 13, 2001;413(6852):174-9).

Ephrin ligands are highly expressed in central nervous system (CNS) germinal regions (Conover et al., 2000, Nature Neurosci., Vol. 3, Issue 11, 1091-1097; Stuckmann et al., 2001, J. Neurosci., Vol. 21, Issue 8, 2726-2737), suggesting that they may be involved in regulating the migration of glial progenitor cells into the surrounding pia and axonal guidance across midline.

Ephs have been shown by applicants to play a role in neurological development through the regulation of cell migration. Through the use of anti-Eph antibodies and PCR, it has been shown that EphB1 is expressed in cultured immature and mature rodent oligodendrocytes and that expression levels of EphB1 decrease as cells mature. These studies also demonstrate that oligodendrocyte migration can be affected by ephrin-B ligands.

The effect of ephrin-B ligands on oligodendrocyte migration was measured using a cell migration assay known as a stripe assay (adapted from Bonhoeffer et al., Development. 1987 December;101(4):685-96). In a typical stripe assay, a putative attractant or repellant is affixed to a plate in a linear shape known as a stripe. A cell suspension is then placed on the plate and allowed to equilibrate. Subsequently, the speed and direction of cell migration relative to the stripe is measured. If the stripe contains a chemorepellant, the cells in the dish will either not migrate into the stripe region or migrate away from the stripe. Therefore, if the cells are observed to avoid the stripe or migrate away from the stripe, then the contents of the stripe are identified as a chemorepellant for that type of cell in the cell culture.

Although Ephs and ephrins interact in vivo via direct cell-cell interaction, it has been shown that linking the extracellular domain of an Eph or an ephrin to an IgG Fc can create a soluble fusion protein capable of activating its respective ephrin or Eph. (Kaneko M, and Nighorn A, J Neurosci. Dec. 17, 2003;23(37):11523-38).

A stripe assay was performed using an ephrin-B-Fc fusion protein capable of activating the EphB1 receptor without requiring cell-cell interaction. The ephrin-B-Fc fusion protein was adhered to a plate in a linear-shaped area known as a stripe. A suspension of oligodendrcytes were then deposited on the plate, and the speed and direction of the migration of the cultured oligodendrocytes was measured relative to the ephrin-B-Fc stripe. It has been shown that a stripe of ephrin-B-Fc repulses the migration of cultured oligodendrocytes in vitro.

In the CNS, the myelin sheath around axons serves as an insulator that increases the speed of signal propagation along the axon. Myelin is produced by oligodendrocytes, and consists of multiple layers of oligodendrocyte membrane wrapped around the axon. In demyelinating diseases such as Multiple Sclerosis (“MS”), neurological symptoms result from impaired conduction in demyelinated axons. Other demyelinating disorders include central pontine myelinolysis, leukodystrophies, acute disseminated encephalomyelitis, progressive multifocal leukoencephalopathy, and subacute sclerosing panencephalitis. Neuropathological examination of the demyelinating MS foci has revealed a pronounced decrease in oligodendrocyte numbers. Loss of oligodendrocytes has been observed in both acute and chronic MS lesions. It is suggested that the reduction of oligodendrocytes in MS foci is the result of oligodendrocyte death (Bruck, W., et al. (1994) Ann Neurol 35, 65-73.).

Ephrin ligands have been shown to directly inhibit oligodendrocyte and neuronal migration. Moreover, the EphB1 receptor and corresponding Ephrin ligands are upregulated under pathological conditions, including Multiple Sclerosis lesions, spinal cord injury, and lung and breast tumors, suggesting involvement of this receptor in restricting cellular migration in diseased tissue ( Bundesen et al., 2003, J. Neurosci., 23 (21), 7789-7800).

Newly formed oligodendrocytes are present in and around MS lesions, suggesting the possibility of self-repair if these cells are able to migrate into the lesions. Recent studies suggest that the migration of these oligodendrocyte progenitor cells may be influenced by expression of EphB1-mediated inhibitory signals. It is further suggested that interfering with the EphB1 signaling pathway may allow oligodendrocyte progenitor cells to migrate into injured brain regions and positively influence repair processes.

Accordingly, it is desirable to identify compounds which interfere with EphB1-mediated cell repulsion. The present invention provides methods for identifying compounds which interfere with the EphB1 signaling pathway by setting forth screening assays for a modulator of EphB1 receptor activity. It is further desirable to use such identified compounds to treat patients suffering from demyelinating disorders such as MS.

### BRIEF SUMMARY OF THE INVENTION

In one aspect, the invention relates to a method of identifying a compound capable of inhibiting EphB1 activity having the steps of: measuring EphB1 activity in the absence of a candidate compound; and measuring EphB1 activity in the presence of the candidate compound, wherein said candidate compound is identified as capable of inhibiting EphB1 activity if the activity measured in the presence of the candidate compound is less than the activity measured in the absence of the candidate compound. In a further aspect of the present invention, EphB1 activity may be measured in one of three assays presented below: a cell repulsion assay, a tyrosine kinase assay, and an in vivo assay.

Cell repulsion may be measured by affixing an ephrin-B ligands to a specific region or regions on a plate, adding a suspension of EphB1 expressing cells to the plate, and measuring the rate, extent, and direction of migration of the cells relative to the specific region or regions. The ephrin-B ligand may be affixed to the plate as an ephrin-B-Fc fusion protein, as a protein expressed on the surface of a cell wherein the cell is affixed to the plate, or as a protein incorporated into a plasma membrane wherein the plasma membrane is affixed to the plate.

EphB1 tyrosine kinase activity is determined by measuring the phosphorylation activity of EphB1's intracellular tyrosine kinase domain. Tyrosine kinase activity may be measured in intact cells.

EphB1 activity may be determined in vivo by measuring the progress of or rate of repair in a demyelinating animal model. The animal model may be an rodent EAE or EtBr-induced lesions.

In another aspect, the invention relates to inhibiting EphB1 activity in a human host by administering a compound that inhibits activity of the EphB1 gene product in a human host in need of such treatment, wherein the ability of the compound to inhibit the activity of the EphB1 gene product is identified by measuring the activity of said EphB1 gene product in the absence of a candidate compound and measuring the activity of said EphB1 gene product in the presence of the candidate compound, wherein the candidate compound is identified as capable of inhibiting EphB1 activity if the activity measured in the presence of the candidate compound is less than the activity measured in the absence of the candidate compound. In a further aspect, the compound is administered as a pharmaceutical composition having the compound and a pharmaceutically-acceptable adjunct.

### BRIEF DESCRIPTION OF THE DRAWINGS

FIG. 1 is a bar chart depicting the relative levels of EphB1 mRNA expression in rat oligodendrocyte progenitor cells (OLP), mature oligodendrocytes (OL), astrocytes, and microglia.

FIG. 2 is a chart depicting the relative levels of EphB1 mRNA expression in a wide variety of human tissue types selected from throughout the body.

FIG. 3 is a chart depicting the relative levels of EphB1 mRNA expression in different subregions of the adult human brain.

FIG. 4 is a chart depicting the relative levels of EphB1 mRNA expression in human pathological tissues.

FIG. 5 is a bar chart depicting the relative levels of EphB1 mRNA expression in human white matter from normal and MS brains. MS tissue samples represent lesions with varying degrees of severity based on histopathological assessment (“MS-PVC”, tissue containing perivascular cusps; “50% plaque”, tissue containing less than or equal to about 50% plaque; “>50% plaque”, tissue containing greater than 50% plaque; “100% plaque”, tissue containing 100% plaque; “MS-NAWM”, tissue containing normal “appearing” white matter; “C-WM”, tissue from normal adult brain).

FIG. 6 is a bar chart depicting the relative levels of EphB1 expression in various tissue types including MS lesions of varying degrees of severity.

### DETAILED DESCRIPTION OF THE INVENTION

A series of EphB1 mRNA expression profiles were performed on a variety of cell types to determine the tissues in which EphB1 mRNA is expressed, as well as the relative levels of expression.

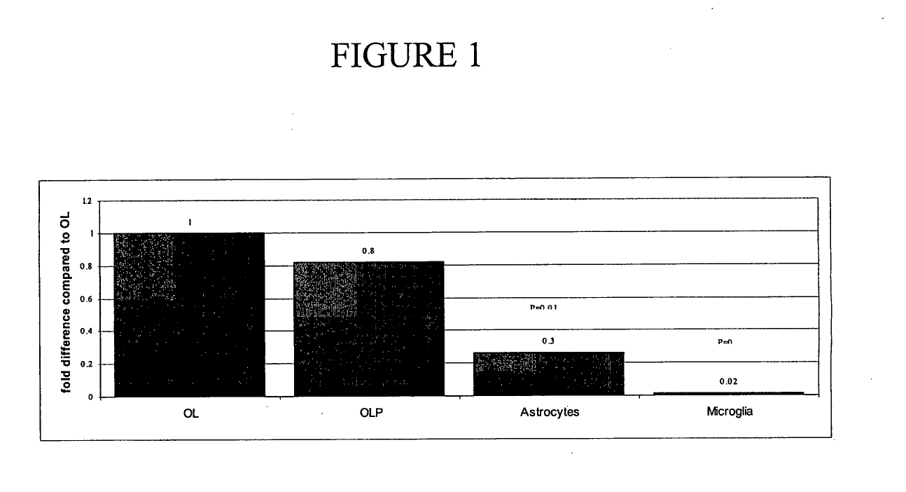

EphB1 mRNA levels were found to be enriched in oligodendrocytes. FIG. 1 is a bar chart depicting the relative levels of EphB1 mRNA expression in four types of cells: mature oligodendrocytes (“OL”), oligodendrocyte progenitor (“OLP”) cells, astrocytes, and microglia. The measurements of EphB1 expression were taken using a real time reverse transcriptase polymerase chain reaction (“RT-PCR”) assay. RT-PCR is a fluorescence-based assay that is well established in the art for the quantification of steady-state mRNA levels. In this RT-PCR assay, EphB1 mRNA is reverse-transcribed and then amplified using PCR. The PCR is performed using specially designed probes containing fluorophores and quenchers such that fluorophores are separated from their quenchers in each round of amplification, with the result that the level of fluorescence increases proportionally to the quantity of amplified EphB1. The number of amplification cycles needed for the fluorescence level to reach a predetermined threshold is measured. The number of amplification cycles needed for the fluorescence level to reach a predetermined threshold is defined as Ct. A number of amplification reactions are performed and several Ct values are measured. The average of these Ct values is then computed. The average Ct value is defined as dCt. A high number of cycles to reach the threshold indicate a low starting quantity of the transcript. Conversely, the fewer the number of cycles needed to reach the threshold, the higher the starting quantity of the transcript. Therefore, mRNA expression is inversely proportional to dCt. Unless otherwise specified, the expression levels depicted in the charts herein were calculated by normalizing the measured mRNA expression level to that of one or more housekeeping genes such as 18S RNA or β2 microglobulin. As shown in FIG. 1, the level of EphB1 mRNA is significantly enriched in rat oligodendrocyte progenitor cells (“OLP”) and mature oligodendrocytes (“OL”) compared to astrocytes and microglia.

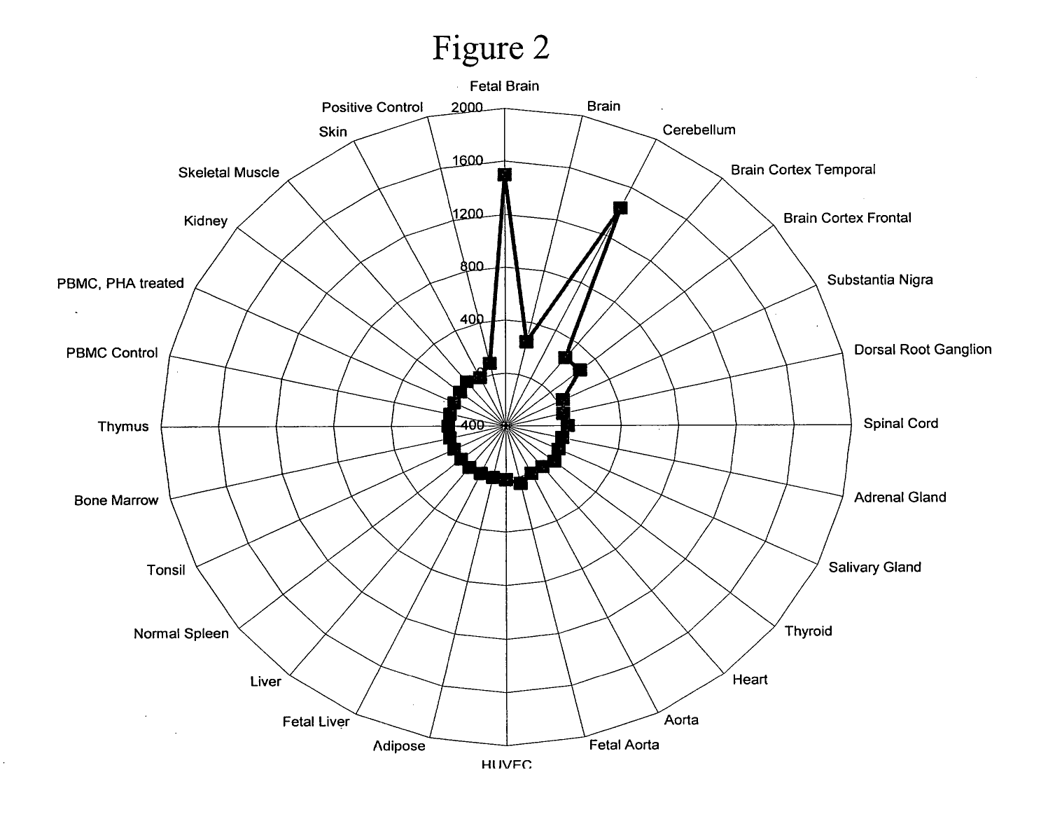

EphB1 mRNA levels were found to be enriched in the human central nervous system (“CNS”). FIG. 2 is a chart depicting the relative levels of EphB1 mRNA expression in a wide variety of tissue types selected from throughout the body. The levels were measured using RT-PCR. As shown in FIG. 2, EphB1 mRNA has relatively high expression levels in fetal and adult brain tissues.

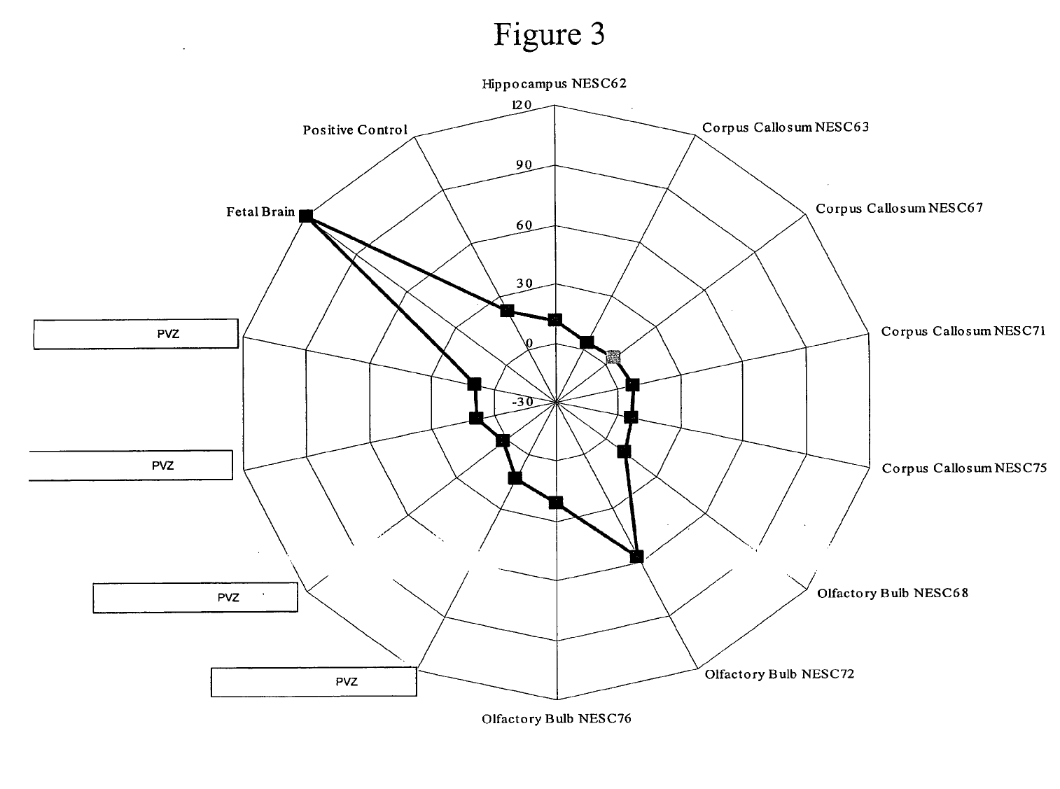

Within the adult CNS, EphB1 mRNA is expressed at a lower level in human adult white matter. FIG. 3 is a chart depicting the relative levels of EphB1 mRNA expression in different subregions of the adult human brain. As shown in FIG. 3, EphB1 mRNA is relatively low in adult human white matter.

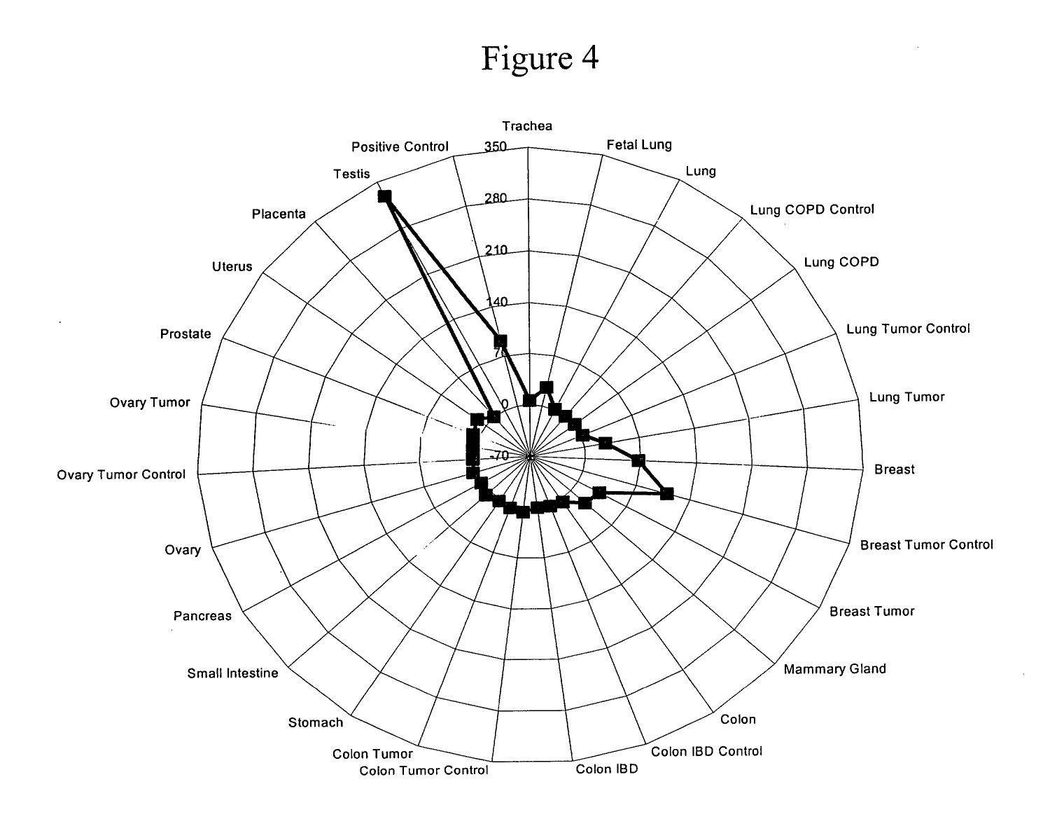

EphB1 mRNA expression levels have been shown to be increased in certain human pathologies. As seen in FIG. 4, EphB1 mRNA levels are increased in human lung and breast tumors. Expression in these tissue types suggests the EphB1 is involved in modulating cellular migration in diseased tissue.

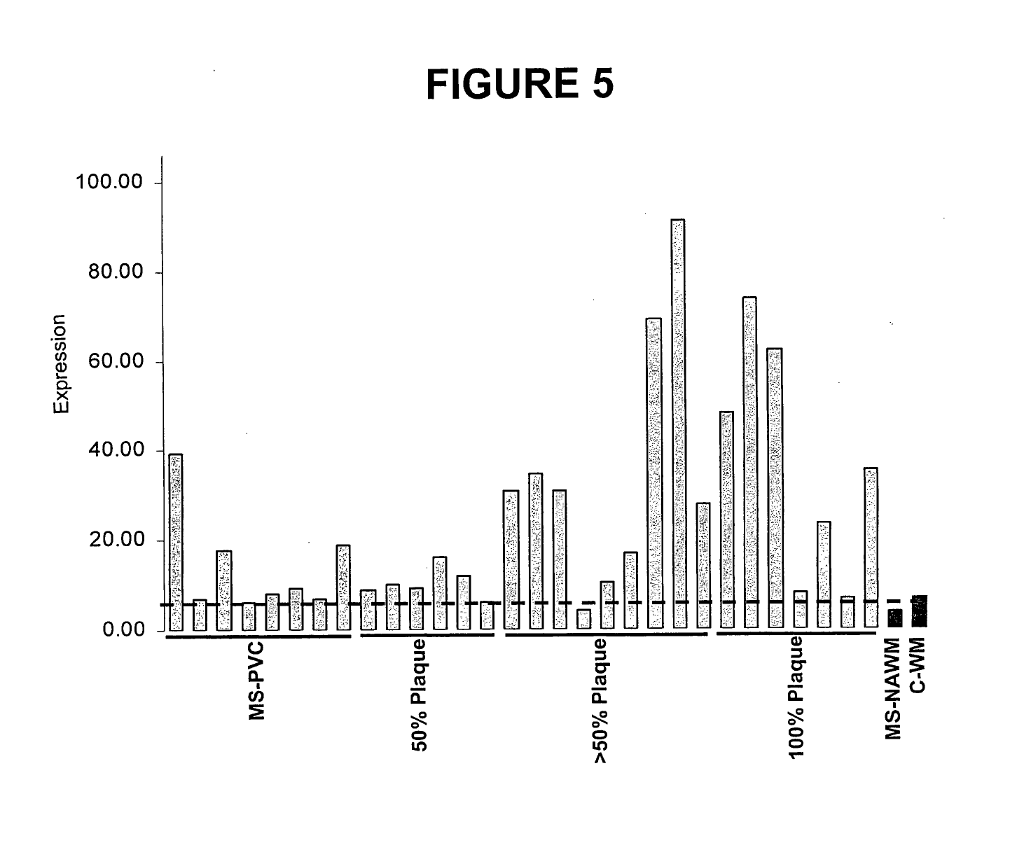

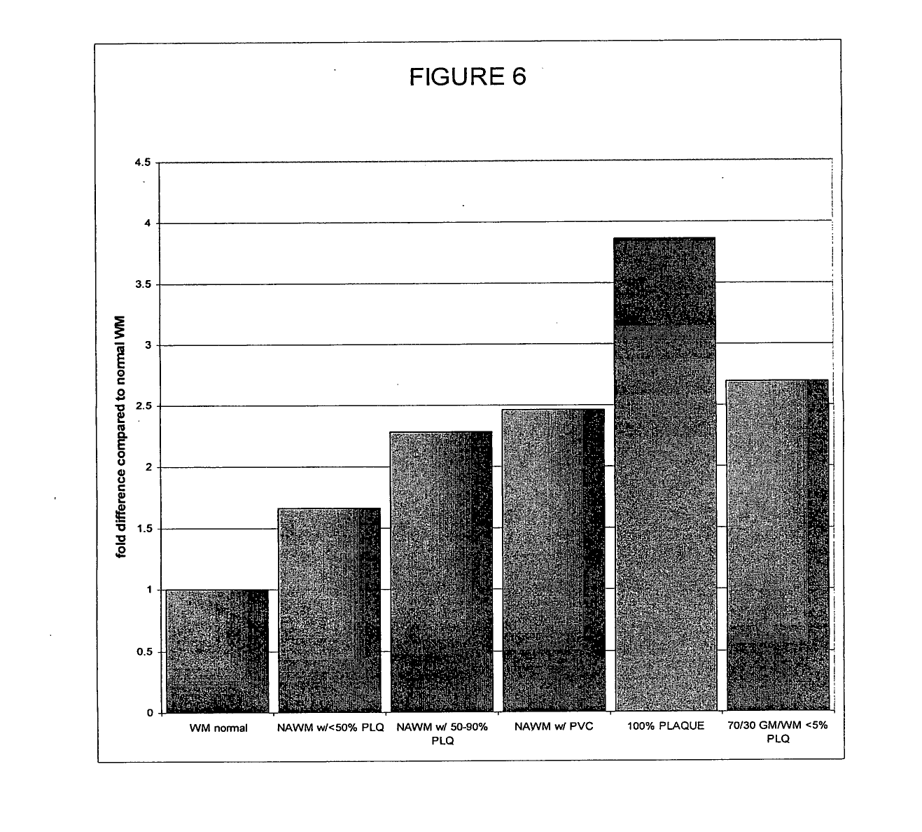

FIG. 5 is a bar chart depicting EphB1 mRNA expression levels in MS lesions of varying severity. As shown in FIG. 5, EphB1 mRNA levels increase as the amount of plaque material is increased, suggesting that are highest in the gray matter of MS lesions. Similarly, FIG. 6 is a bar chart depicting the expression of EphB1 mRNA in MS lesions of varying severity relative to normal white matter. As shown in FIG. 6, EphB1 mRNA expression levels are highest in the most severe MS lesions.

These results indicate that glial progenitor cells expressing EphB1 on their cell surface are subject to increased ephrin-B-mediated cell repulsion in and around MS plaques, with the result that their ability to migrate into regions of inflammation and demyelination may be significantly impaired or prevented. If this ephrin-B-mediated cell repulsion is blocked, then it is expected that increased numbers of oligodendrocyte progenitor cells will migrate into the MS-lesions where they can interact with axons, differentiate, and reform myelin sheaths.

One method of interfering with ephrin-B-mediated cell repulsion of EphB1-expressing cells such as oligodendrocytes involves identifying a compound capable of interfering either with the interaction between the EphB1 receptor and an ephrin-B ligand, or with the function of EphB1, specifically the EphB1 signaling pathway. Such an identified compound could then be administered to a patient suffering from a demyelinating disorder such as multiple sclerosis. Other demyelinating disorders include central pontine myelinolysis, leukodystrophies, acute disseminated encephalomyelitis, progressive multifocal leukoencephalopathy, and subacute sclerosing panencephalitis.

In one embodiment of the invention, a compound is identified as capable of interfering with ephrin-B-mediated cell repulsion by measuring the rate of such repulsion in the presence and absence of a candidate compound.

One assay used to measure the rate of cell repulsion is known as a stripe assay. Bonhoeffer et al., Development. 1987 December;101(4):685-96. For the purposes of this invention, a “stripe assay” is a cell migration assay performed in vitro in which a putative attractant or repellant is affixed to a plate in one or more linear shaped regions known as a stripes, and wherein a cell culture is placed on the plate and the speed and direction of the cells' migration is measured relative to the stripe or stripes.

A typical stripe assay employs one linear-shaped region, known as a stripe, containing a putative repulsor molecule adhered to a plate, along with a cell suspension plated adjacent to the stripe. The extent, speed, and/or direction of cell migration is then measured using real time photoimaging techniques. If the stripe contains a chemorepellant, the cells will either avoid the stripe or migrate away from the stripe. Another variant of the stripe assay employs a series of parallel linear-shaped regions, known as stripes, of the putative repulsor molecule, separated by a known distance called a gap. In this variant, if the stripes contain a repellant, the cells in the cell culture will either avoid or migrate away from the stripes and into the gaps between the stripes.

In one embodiment of the stripe assay, a stripe or stripes comprising an adhered ephrin-B-Fc fusion protein is employed. Although Ephs and ephrins interact in vivo via direct cell-cell interaction, it has been shown that linking the extracellular domain of an Eph or an ephrin to an IgG Fc can create a soluble fusion protein capable of activating its respective ephrin or Eph. (Kaneko M, and Nighorn A, J Neurosci. Dec. 17, 2003;23(37): 11523-38). An ephrin-B-Fc fusion protein comprises a functional portion of an ephrin-B receptor operatively linked to the Fc region of an IgG immunoglobulin. Construction of a suitable ephrin-B-Fc fusion protein is described in Beckmann, M. P., et al., EMBO J. 13: 3757-3762 (1994) and Davis, S. et al., Science 266, 816-819 (1994). In an alternate embodiment, the stripe or stripes comprise an affixed cell membrane comprising ephrin-B ligands. In yet another embodiment, the stripe or stripes comprise adhered cells expressing an ephrin-B ligand on their surface.

In a modification of the above-referenced assay, two sets of stripe assays are performed measuring the extent, rate, and direction of cell migration of EphB1-expressing cells relative to the stripe or stripes. In the first set of assays, migration is measured in the absence of a candidate compound. In the second set, the compound is added and migration is measured in the presence of the candidate compound. The extent, rate, and direction of migration is then compared between the two sets of assays. A candidate compound is identified as a compound capable of interfering with ephrin-B-mediated cell repulsion if the measured extent of migration onto the stripe is increased or the extent or rate of cell migration away from the stripe or stripes is lower in its presence than in its absence. Similarly, a candidate compound is identified as a compound capable of interfering with ephrin-B-mediated cell repulsion if the measured extent or direction of cell migration into the stripe is higher or away from the stripe or stripes is lower in its presence than in its absence. In an alternate embodiment, the cell migration assay uses a repulsor molecule affixed to a plate in a shape other than a linear shape. For example, the repulsor can be affixed to the plate at one specific point.

Another approach to reduce ephrin-B-mediated cell repulsion of EphB1-expressing cells is to interfere with the function of EphB1 by modulating its signaling pathway. The EphB1 protein contains an intracellular tyrosine kinase domain involved in signal transduction. (Vindis, et al., J Cell Biol. Aug. 18, 2003;162(4):661-71). The intracellular tyrosine kinase domain of EphB1 is located at positions 613 to 881 of SEQ ID NO:1. Interfering with this tyrosine kinase domain's function will prevent signaling along the EphB1 pathway and thus attenuate ephrin-B-mediated cell repulsion.

Thus, in another embodiment of the invention, a compound is identified as capable of interfering with the tyrosine kinase activity of EphB1's intracellular tyrosine kinase domain by measuring EphB1-mediated tyrosine kinase activity in the presence and absence of a candidate compound. In this embodiment, two sets of tyrosine kinase assays are performed. The first set is performed in the absence of the candidate compound. In the second set, the compound is added and activity is measured in the presence of the compound. Tyrosine kinase activity is then compared between the two sets of assays. A candidate compound is identified as a compound capable of interfering with the tyrosine kinase activity of EphB1's intracellular tyrosine kinase domain if the measured tyrosine kinase activity is significantly lower in its presence than in its absence.

Methods of measuring tyrosine kinase activity are well established in the art. For example, tyrosine kinase assay kits are available commercially from Roche Molecular Biosystems, Calbiochem, Chemicon, Perkin-Elmer Life Sciences, Upstate Biotechnologies, and Applied Biosystems. The tyrosine kinase assay may employ a substrate peptide comprising a fluorescent tag and an antibody specific to phosphorylated tyrosine that is affixed to a surface such as a bead or a well. As the substrate peptide gets phosphorylated, it binds to the antibody and thus it and its fluorescent tag are localized to where the antibody is attached. If the substrate does not get phosphorylated, then the substrate and its fluorescent tag remain diffuse. The level of tyrosine kinase activity can be measured by determining the level of fluorescence at the location where the antibody is attached.

Tyrosine kinase activity may also be measured in intact EphB1-expressing cells or in small plasma membrane vesicles comprising EphB1 protein on their surface. These vesicles may be created by sonicating intact EphB1-expressing cells. Tyrosine kinase activity may also be measured using a cell lysate of EphB1-expressing cells, or using isolated fragments of EphB1 comprising its intracellular domain. Recombinantly made EphB1 intracellular tyrosine kinase domain may also be used to carry out the above invention. In this embodiment, the DNA sequence of EphB1's tyrosine kinase domain (nucleotides 2051 to 2857 of SEQ ID NO:2) is cloned into an expression vector such as the pMAL vector available from New England Biolabs, which is then expressed in E. coli cells and purified according to the pMAL System protocol. (New England Biolabs pMAL Protein Fusion and Purification System Manual, available from New England Biolabs.)

EphB1 activity may be measured by measuring the activity of other elements on its signaling pathway. Known elements downstream of EphB1 include Cdc42 and Rac. (Murai and Pasquale, Journal of Cell Science 116, 2823-2832 (2003)). Cdc42 and Rac are GTPases whose activity may be measured by measuring the amount of label released from a labelled GTP substrate or by measuring fluorescence resonance energy transfer (“FRET”) assay described in Kraynov, V. S., et al., Science 290:333-337 (2000). A typical FRET assay measures the release of a fluorophore from a substrate which has been microinjected into intact cells. In this embodiment, activity of a downstream element is measured in two sets of assays. The first set is performed in the absence of the candidate compound. In the second set, the compound is added and activity is measured in the presence of the compound. Downstream element activity is then compared between the two sets of assays. A candidate compound is identified as a compound capable of interfering with the tyrosine kinase activity of EphB1's intracellular tyrosine kinase domain if the measured tyrosine kinase activity is significantly lower in its presence than in its absence.

In yet another embodiment of the invention, a candidate compound's effect on ephrin-B mediated cell repulsion is measured in vivo in an animal models of demyelination and remyelination, including the mammalian ethidium bromide (“EtBr”) and experimental autoimmune encephalomyelitis (“EAE”) models in rat, mouse, and marmoset. In this embodiment, “MS”-like symptoms and pathophysiology are induced in the animal. The animal is then treated with a candidate compound. The progress of MS in the animal is monitored. Typically, the progress of MS in an animal model is quantified as a number known as a “clinical score,” which typically ranges on a scale of zero (healthy) to five (moribund or dead) based on the severity of MS symptoms in the animal. At specific time points, animals are sacrificed and evaluated for remyelination by LUXOL FAST BLUE (“LFB”) and myelin basic protein (“MBP”) staining to confirm remyelination. A candidate compound is identified as capable of interfering with ephrin-B-mediated cell repulsion if treated animals show significantly improved clinical scores or remyelination over that of an untreated animal. Alternatively, compound efficacy may increase the rate and/or extent of remyelination over that of untreated animals.

Examples of candidate compounds include, but are not limited to, a small molecule such as is generated by a combinatorial chemistry process, or a macromolecule such as a protein, nucleic acid, carbohydrate, lipid, glycoprotein, lipoprotein, polysaccharide, any modified derivative of the above molecules, as well as any complex comprising one or more of the above molecules. One example of a candidate compound is a double-stranded RNA used for RNA interference (“RNAi”). RNAi is a method of inhibiting expression of a target gene described in detail, for example, in U.S. Pat. No. 6,506,559. RNAi methods and materials are further described in U.S. Patent Application Publication Nos. 20020086356 and 20030108923, and an overview of RNAi is provided in Tuschl, Chembiochem. 2;2(4):239-45 (April, 2001).

A compound identified by the foregoing methods may be administered alone or in the form of a pharmaceutical composition in combination with pharmaceutically acceptable carriers or excipients. An identified compound may be administered in any form or mode that makes the compound bioavailable in effective amounts. Identified compounds may be administered orally, subcutaneously, intramuscularly, intravenously, transdermally, intranasally, rectally, ocularly and the like. Oral administration is preferred. A pharmaceutical composition of an identified compound may be adapted for the route of administration. Examples of pharmaceutical compositions of an identified compound include a tablet, troche, capsule, elixir, syrup, wafer, chewing gum, suppository, solution or suspension if the route of administration is oral, parental or topical. A preferred oral pharmaceutical composition of an identified compound comprises the compound with an inert diluent or with an edible carrier. One skilled in the art of preparing pharmaceutical formulations may readily determine appropriate forms of an identified compound by determining particular characteristics of the compound, the disease to be treated, the stage of the disease, response of other patients and other relevant circumstances.

It may be desirable-to administer an identified compound to the brain. Examples of methods for administering an identified compound to the brain include, but are not limited to local infusion during surgery, injection, use of a catheter, use of a suppository, or use of an implant. Implants can comprise a porous, non-porous, or gelatinous material, including membranes, such as sialastic membranes or fibers. When it is desirable to direct the drug to the central nervous system, techniques which can opportunistically open the blood-brain barrier for a time adequate to deliver the drug there through can be used. For example, a composition of 5% mannitose and water can be used.

It is anticipated that pharmaceutical compositions of the present invention will be administered periodically, both during active episodes of disease and during periods of remission, either alone or in conjunction with one or more anti-inflammatory agents. It is anticipated that the pharmaceutical compositions of this invention, when properly administered, will allow the migration of EphB1-expressing cells such as oligodendrocyte precursor cells to migrate into diseased loci expressing cell repulsion effectors such as ephrin-B ligands. It is further anticipated that materials and methods of this invention can be used to treat other pathologies capable of amelioration by attenuating cell repulsion, including demyelination-related pathologies.

What is claimed is:

1. A method of identifying a compound capable of inhibiting ephrin-B-mediated EphB1 activity comprising the steps of:
(a) measuring the activity of said EphB1 in the absence of a candidate compound; and
(b) measuring the activity of said EphB1 in the presence of said candidate compound,
wherein said candidate compound is identified as capable of inhibiting ephrin-B-meditated EphB1 activity if the activity measured in step (b) is less than the activity measured in step (a).

2. The method of claim 1 wherein said measuring step (a) comprises measuring the ephrin-B-mediated repulsion of an EphB1-expressing cell in the absence of a candidate compound, and said measuring step (b) comprises measuring the ephrin-B-mediated repulsion of said cell in the presence of said candidate compound.

3. The method of claim 2 wherein said measuring of ephrin-B-mediated cell repulsion comprises the steps of:
(i) affixing an ephrin-B ligand to at least one specific region on a plate;
(ii) adding a cell culture expressing EphB1 to said plate; and
(iii) measuring the extent, speed, and direction of migration of said cell culture relative to said at least one specific region.

4. The method of claim 3 wherein said ephrin-B ligand is an ephrin-B-Fc fusion protein.

5. The method of claim 3 wherein said ephrin-B ligand is affixed to said plate by means of an ephrin-B-expressing cell.

6. The method of claim 3 wherein said ephrin-B ligand is affixed to said plate by means of a plasma membrane.

7. The method of claim 1 wherein said measuring step (a) comprises measuring the kinase activity of the intracellular tyrosine kinase domain of said EphB1 in the absence of said candidate compound, and said measuring step (b) comprises measuring the kinase activity of the intracellular tyrosine kinase domain of said EphB1 in the presence of said candidate compound.

8. The method of claim 7 wherein said measuring of the activity of EphB1's intracellular tyrosine kinase domain measures the tyrosine kinase activity of an intact cell.

9. The method of claim 1 wherein said measuring step (a) comprises measuring the progress of a demyelinating disorder in an animal in the absence of said candidate compound, and said measuring step (b) comprises measuring the progress of a demyelinating disorder or rate/extent of repair in an animal in the presence of said candidate compound.

10. The method of claim 9, wherein said animal is selected from the group consisting of:
an experimental autoimmune encephalomyelitis (“EAE”), and an ethidium bromide (“EtBr”) model.

11. A method for inhibiting EphB1 activity in a human host, comprising administering a compound that inhibits activity of the EphB1 gene product in a human host in need of such treatment, wherein the ability of the compound to inhibit the activity of the EphB1 gene product is identified by:
(a) measuring the activity of said EphB1 gene product in the absence of a candidate compound; and
(b) measuring the activity of said EphB1 gene product in the presence of said candidate compound,
wherein said candidate compound is identified as capable of inhibiting EphB1 activity if the activity measured in step (b) is less than the activity measured in step (a).

12. The method of claim 11 wherein said compound is administered as a pharmaceutical composition comprising said compound and a pharmaceutically-acceptable adjunct.

13. The method of claim 11 wherein said measuring step (a) comprises measuring the ephrin-B-mediated repulsion of an EphB1-expressing cell in the absence of a candidate compound, and said measuring step (b) comprises measuring the ephrin-B-mediated repulsion of said cell in the presence of said candidate compound.

14. The method of claim 13 wherein said measuring of ephrin-B-mediated cell repulsion comprises the steps of:
(i) affixing an ephrin-B ligand to at least one specific region on a plate;
(ii) adding a cell culture expressing EphB1 to said plate; and
(iii) measuring the extent, speed, and direction of migration of said cell culture relative to said at least one specific region.

15. The method of claim 14 wherein said ephrin-B ligand is an ephrin-B-Fc fusion protein.

16. The method of claim 14 wherein said ephrin-B ligand is affixed to said plate by means of an ephrin-B-expressing cell.

17. The method of claim 14 wherein said ephrin-B ligand is affixed to said plate by means of a plasma membrane.

18. The method of claim 11 wherein said measuring step (a) comprises measuring the kinase activity of the intracellular tyrosine kinase domain of said EphB1 in the absence of said candidate compound, and said measuring step (b) comprises measuring the kinase activity of the intracellular tyrosine kinase domain of said EphB1 in the presence of said candidate compound.

19. The method of claim 18 wherein said measuring of the activity of EphB1's intracellular tyrosine kinase domain measures the tyrosine kinase activity of an intact cell.

20. The method of claim 11 wherein said measuring step (a) comprises measuring the progress of a demyelinating disorder in an animal in the absence of said candidate compound, and said measuring step (b) comprises measuring the progress of a demyelinating disorder or extent/rate of repair in an animal in the presence of said candidate compound.

21. The method of claim 20, wherein said animal is selected from the group consisting of: an experimental autoimmune encephalomyelitis (“EAE”), and an ethidium bromide (“EtBr”) models.
